(19) United States
(12) Patent Application Publication
(10) Pub. No.: US 2008/0026078 A1
(43) Pub. Date: Jan. 31, 2008

(54) NASAL COMPOSITION

(76) Inventors: James ZINREICH, Baltimore, MD (US); Eva ZINREICH, Baltimore, MD (US); Helen Zinreich SHAFER, Baltimore, MD (US)

Correspondence Address: HAHN LOESER & PARKS, LLP, One GOJO Plaza, Suite 300, AKRON, OH, 44311-1076, US

(21) Appl. No.: 11/782,959

(22) Filed: Jul. 25, 2007

Related U.S. Application Data
(60) Provisional application No. 60/820,289, filed on Jul. 25, 2006.

(51) Int. Cl. A61K 33/14; A61K 31/375

(52) U.S. Cl. 424/680; 514/474

(57) ABSTRACT

A nasal spray solution for use as a nasal spray and methods of using the solution are disclosed. The nasal spray solution moisturizes nasal passages and alleviates nasal dryness. The solution includes a hypotonic saline solution, wherein the saline solution is hypotonic with respect to cells of the nasal mucosa of the nasal passages, a buffering agent, a lubricating agent, and an anti-microbial agent.

This U.S. patent application claims priority to U.S. Provisional Application Ser. No. 60/820,289, filed Jul. 25, 2006, and is hereby incorporated by reference.

### TECHNICAL FIELD

The invention relates to a saline solution for a nasal spray that moisturizes nasal passages and alleviates nasal dryness and methods of use. In particular, the invention relates to a hypotonic or isotonic saline solution for a nasal spray, which includes a lubricating agent and an anti-microbial agent that moisturizes nasal passages and lessens or alleviates nasal dryness.

### BACKGROUND OF THE INVENTION

The nose is a complicated structure that serves dual functions as the organ for the sense of smell and as an entry to the respiratory tract. As part of the respiratory tract, a healthy nose moisturizes and warms incoming air and filters out foreign materials.

Nasal passages and other portions of the respiratory tract are lined with specialized tissue layers. In the nose and sinus areas this tissue is often called the nasal mucosa. Like many tissues, the nasal mucosa is composed of several cell layers and cell types. Mucous cells are one type of cell found in the nasal mucosa. These cells are found throughout the nasal mucosa and are generally clustered into small glands. These glands secrete a sticky substance called mucus. Mucus is composed of water, shed epithelial (surface) cells, dead leukocytes, mucin, and inorganic salts, among other things, that are all held in suspension. Mucus functions as a trap for airborne particles (e.g., dust, bacteria, and viruses) that enter the nasal passages. Mucus also lubricates the walls of the nose, sinuses, and throat.

In a healthy nose, the mucus is cleared from the nasal passages on a regular basis by a layer of cells in the nasal mucosa called the ciliated columnar epithelium. These cells possess small hair-like projections called cilia that undulate and “sweep” mucus through the nasal passages allowing it to drain to the back of the throat where it can be swallowed or expelled. This line of defense protects the body against the bacteria and viruses that continually enter the nose and mouth.

Connected to the nose are sinuses or air-filled cavities located behind certain facial bones. There are four groups of sinuses, namely, frontal, sphenoidal, ethmoidal, and maxillary. The sinuses are also lined with mucus secreting tissue. The sinuses are normally kept clear when mucus drains through them into the nasal passages. However, under low humidity atmospheric conditions typically found on airplanes and in desert areas, the sinuses can become dry resulting in nasal discomfort.

Nasal sprays have been commercially available for moisturization of the nasal membranes. Such sprays normally are water containing surfactants to spread the water over the nasal membranes and enhance the penetration of the water into the surface layers of the membranes. There is no therapeutic function of the water penetration.

Therefore, it would be advantageous to provide a hypotonic or isotonic saline solution for a nasal spray or nasal drop solution that includes a lubricating agent and an anti-microbial agent for moisturizing the nasal passages and lessening or alleviating nasal dryness.

### SUMMARY OF THE INVENTION

An embodiment of the invention is directed to a solution for a nasal spray or nasal drops that moisturize the nasal passages and lessens or alleviates nasal dryness. The solution includes a hypotonic saline solution, wherein the saline solution is hypotonic with respect to cells of the nasal mucosa of the nasal passages, a buffering agent, a lubricating agent, and an anti-microbial agent.

Another embodiment of the invention is directed to a method of moisturizing nasal passages and alleviating nasal dryness. The method includes the steps of providing a nasal spray solution, wherein the solution includes a hypotonic saline solution, wherein the saline solution is hypotonic with respect to cells of the nasal mucosa of the nasal passages, and delivering at least one application of the solution into a nostril, wherein the solution moisturizes the nasal mucous membrane of the nasal passages. The method further includes a buffering agent, a lubricating agent, and an anti-microbial agent.

In yet a further embodiment of the invention, a nasal spray solution for moisturizing nasal passages and alleviating nasal dryness is disclosed. The solution includes a hypotonic saline solution, wherein the saline solution is in a range from about 0.4% to about 0.6% by weight sodium chloride and hypotonic with respect to cells of the nasal mucosa of the nasal passages, a buffering agent, a lubricating agent, wherein the lubricating agent is in a range from about 0.5% to about 1.0% by volume of the composition of the solution, and an anti-microbial agent, wherein the anti-microbial agent is in a range from about 0.1% to about 0.5% by volume of the composition of the solution.

This and other objects of the invention are achieved by the hypotonic or isotonic saline solution found in the spray liquid for the nasal spray used for moisturizing the nasal passages to alleviate nasal dryness, as described in more detail below.

### DETAILED DESCRIPTION OF THE INVENTION

In general, a solution for a nasal spray that moisturizes and provides anti-microbial activity towards airborne diseases when applied into nasal passages is disclosed. The solution is a hypotonic or isotonic nasal spray or nasal drops that aid in the moisturizing, in offering some localized anti-microbial activity and increased ciliary function.

For purposes of this discussion, osmosis may be defined as the spontaneous passage or diffusion of water through a semipermeable membrane which is a membrane that is capable of blocking the passage of dissolved substances from an area of higher water concentration to an area of lower water concentration. Stated alternatively, water will diffuse upwardly towards its concentration gradient or into a hypotonic environment to achieve a state of equilibrium. In an isotonic environment, water will essentially be maintained in its environment since a state of equilibrium has been achieved. The semipermeable membranes at issue here are the tissues of the nasal mucosa and even the plasma membranes of individual cells within the tissues.

In an embodiment of the invention, the solution for a nasal spray is a hypotonic or isotonic saline solution. In particular, the saline solution is hypotonic with respect to the tissue and cells of the nasal mucosa. A hypotonic solution has less than an amount of dissolved solute in it when compared to its surroundings and an isotonic solution has about an equal amount of dissolved solute in it when compared to its surroundings. Accordingly, in one embodiment of the invention, the saline solution is equal to or less than about 0.9% by weight (i.e., equal to or less than about 9.0 mg/ml) salt in an aqueous solution. In one embodiment, the saline solution is in a range from about 0.4% to about 0.6% by weight of sodium chloride or sodium chloride equivalent in water (i.e. in the range from about 4.0 mg/ml to about 6.0 mg/ml).

As noted above, the composition according to the invention uses osmosis, with the use of the hypotonic or isotonic saline solution, to aid in the physiological increase of ciliary action by way of increasing the mucociliary clearance in the nasal passages. In general terms with the hypotonic saline solution, the osmotic effect utilized in the invention can be described as follows: by bathing the nasal mucosa in a hypotonic solution, osmotic forces will drive or pull water into the free spaces in the nasal mucosa tissue and into the intracellular environment from the nasal passages thereby hydrating the nasal mucosa, thus providing a moisturizing effect. Furthermore, in general terms with the isotonic saline solution, the osmotic effect utilized in the invention can be described as follows: by bathing the nasal mucosa in an isotonic solution, osmotic forces, since the concentration of the solute in the solution is substantially the same as the concentration in the environment, will essentially allow water to diffuse between and into the free spaces in the nasal mucosa tissue and the intracellular environment from the nasal passages thereby hydrating the nasal mucosa, thus providing a moisturizing effect. The part of the hypotonic or isotonic solution not participating in the osmotic effect, primarily water, will further moisturize the nasal passages and provide an increase in mucociliary clearance of the mucus from the nasal passages.

This osmotic effect requires that the composition be hypotonic or isotonic with respect to the nasal mucosa. Although some of the components in the composition contribute to the hypotonicity or isotonicity of the composition, as used herein, the term osmotic agent refers to an agent specifically added to the composition to decrease the solute level in the composition and contribute to achieving hypotonicity of the spray liquid or nasal drops. In an embodiment of the invention, the osmotic agent used in the practice of the invention is the saline solution previously described.

Besides water, the spray liquid can also include pharmaceutically acceptable additives. The additives, as used herein, include pharmaceutically acceptable carriers, excipients, buffering agents or stabilizers which are nontoxic to the cell or mammal being exposed thereto at the dosages and concentrations employed. Often the pharmaceutically acceptable additive is an aqueous pH buffered solution. Examples of physiologically acceptable carriers include buffers such as, but not limited to, phosphate, borate, citrate and other organic acids; carbohydrates including glucose, mannose, or dextrins; chelating agents such as EDTA; and sugar alcohols such as mannitol.

In yet another embodiment of the invention, the solution of the nasal spray contains an effective amount of an anti-microbial, for example vitamin C or ascorbic acid, to provide an increased localized antibacterial effect. The vitamin C is present in a concentration from about 0.1% to about 0.5% by volume. It has been determined that the anti-microbial agent may also acts as a mucolytic agent. In this capacity, the mucolytic agent performs as a mucociliary stimulant that assists in and increases the breakup and clearance of mucous. The action of cilia in cells of the nasal mucosa is important in keeping the nasal passages clear of mucus. If cilia function is subnormal, mucus will build up and contribute to congestion of the nasal passages.

A preservative may also be optionally used to maintain the integrity of the composition. Suitable preservatives are well known to those skilled in the art and include sorbates, benzoates and mixtures thereof. However, a small quantity (e.g., less than 1% by volume) of potassium sorbate, potassium benzoate or mixtures thereof may be added to the composition.

In a further embodiment of the invention, the liquid spray is a hypotonic or isotonic composition for the use in osmotic moisturization and mucociliary clearance in the nasal passages. The composition is a hypotonic or isotonic saline solution that includes water, sodium chloride, equal to or less than about 0.9% by weight, vitamin C, in the range from about 0.1% to about 0.5% by volume, and glycerol, alone or in combination with sorbitol, in the range from about 0.5% to about 1.0% by volume. In one embodiment, the liquid spray is pH buffered with the addition of mono- and/or di-basic sodium phosphate buffers until a desired physiological pH is achieved.

The nasal spray of the invention is used to moisten the nasal passages in order to alleviate nasal dryness, provide anti-microbial activity and increase mucociliary clearance. Under these circumstances, at least one spray application per nostril will clean and moisturize the nasal mucous membrane. This accomplishes an improved nasal comfortness and well-being by providing a lasting preventative care with respect to illnesses of the respiratory tracts.

Based on the foregoing disclosure, it should be apparent that the liquid spray for a nasal spray of the invention will achieve the objectives set forth above. It is therefore understood that any evident variations will fall within the scope of the claimed invention. Thus, alternate specific component elements can be selected without departing from the spirit of the invention disclosed and described herein.

What is claimed is:

1. A nasal spray solution for moisturizing nasal passages and alleviating nasal dryness, the solution comprising:
a hypotonic saline solution, wherein the saline solution is hypotonic with respect to cells of the nasal mucosa of the nasal passages;
a buffering agent;
a lubricating agent; and
an anti-microbial agent.

2. The solution of claim 1, wherein the saline solution is less than 0.9% by weight sodium chloride.

3. The solution of claim 2, wherein the range of sodium chloride is in a range from about 0.4% to about 0.6% by weight.

4. The solution of claim 1, wherein where the lubricating agent is glycerol, sorbitol or mixtures thereof.

5. The solution of claim 1, wherein the anti-microbial agent is ascorbic acid.

6. The solution of claim 1, wherein the anti-microbial agent is also a mucolytic agent providing mucociliary clearance in the nasal passages.

7. The solution of claim 1, wherein the anti-microbial agent is present in a range from about 0.1% to about 0.5% by volume of the composition of the solution.

8. The solution of claim 1, wherein the lubricating agent is present in a range from about 0.5% to about 1.0% by volume of the composition of the solution.

9. A method of moisturizing nasal passages and alleviating nasal dryness, the method comprising the steps of:
providing a nasal spray solution, the solution comprising a hypotonic saline solution, wherein the saline solution is hypotonic with respect to cells of the nasal mucosa of the nasal passages; and
delivering at least one application of the solution into at least one nostril, wherein the solution moisturizes the nasal mucous membrane of the nasal passages.

10. The method of claim 9, wherein the nasal spray solution further comprises:
a buffering agent;
a lubricating agent; and
an anti-microbial agent.

11. The method of claim 9, wherein the at least one application is sprayed into the at least one nostril.

12. The method of claim 9, wherein the saline solution is less than about 0.9% by weight sodium chloride.

13. The method of claim 12, wherein the range of sodium chloride is in a range from about 0.4% to about 0.6% by weight.

14. The method of claim 9, wherein where the lubricating agent is glycerol, sorbitol or mixtures thereof.

15. The method of claim 9, wherein the anti-microbial agent is ascorbic acid.

16. The method of claim 9, wherein the anti-microbial agent is also a mucolytic agent capable of providing mucociliary clearance in the nasal passages.

17. The method of claim 9, wherein the anti-microbial agent is in a range from about 0.1% to about 0.5% by volume of the composition of the solution.

18. The method of claim 9, wherein the lubricating agent is in a range from about 0.5% to about 1.0% by volume of the composition of the solution.

19. A nasal spray solution for moisturizing nasal passages and alleviating nasal dryness, the solution comprising:
a hypotonic saline solution, wherein the saline solution is in a range from about 0.4% to about 0.6% by weight sodium chloride and hypotonic with respect to cells of the nasal mucosa of the nasal passages;
a buffering agent;
a lubricating agent, wherein the lubricating agent is in a range from about 0.5% to about 1.0% by volume of the composition of the solution; and
an anti-microbial agent, wherein the anti-microbial agent is in a range from about 0.1% to about 0.5% by volume of the composition of the solution.

20. The solution of claim 19, wherein the anti-microbial agent is ascorbic acid.
